(19) United States
(12) Patent Application Publication
(10) Pub. No.: US 2020/0197354 A1
(43) Pub. Date: Jun. 25, 2020

(54) METHODS, COMPOSITIONS AND CONTAINERS FOR REDUCING SOLID FORM QUERCETIN DEGRADATION AND 2,4,6-TRIHYDROXYBENZOIC ACID TOXIC BYPRODUCT THEREOF

(71) Applicant: PUBLIC JOINT STOCK COMPANY "SCIENTIFIC INDUSTRIAL CENTRE "BORSHCHAHIVSKIY CHEMICAL- PHARMACEUTICAL P, Kiev (UA)

(72) Inventors: Sergii Fesenko, Kiev (UA); Alexander Shneider, Dedham, MA (US); Vladimir Gabai, Brighton, MA (US)

(21) Appl. No.: 16/226,743

(22) Filed: Dec. 20, 2018

(51) Int. Cl. A61K 31/352; A61K 9/19; A61K 47/32; A61K 47/02

(52) U.S. Cl. CPC A61K 31/352; A61K 47/02; A61K 47/32; A61K 9/19

(57) ABSTRACT

Provided herein are compositions and methods which reduce degradation of solid form quercetin compositions and reduce the formation of a toxic compound, 2,4,6-trihydroxybenzoic acid (2,4,6-THBA). Also provided are containers and kits that contain solid form quercetin compositions with reduced degradation of quercetin and reduced formation of 2,4,6-THBA. The provided composition and methods increase the shelf life and patient safety of solid form quercetin compositions.

### FIELD OF INVENTION

The invention relates to methods and compositions for the prevention of degradation of quercetin and reduction of the formation of a toxic product, 2,4,6-trihydroxybenzoic acid (2,4,6-THBA). The invention also includes related storage containers. More specifically, the invention relates to the storage of lyophilized quercetin compositions stored in a non-reactive gas atmosphere at ambient temperature.

### BACKGROUND OF THE INVENTION

Quercetin is a plant flavonoid whose inclusion in human diet has been widely associated with a number of health benefits. These benefits include: 1) antioxidant; 2) anti-inflammatory; 3) antiviral; and 4) anticancer activities (Wang et al., 2016). Quercetin is also used to ease cardiovascular diseases (i.e., heart disease, hypertension, and high blood cholesterol).

The bioavailability of quercetin in humans is low and highly variable (0-50%), and it is rapidly cleared with an elimination half-life of 1-2 hours after ingestion in foods or supplements (Graefe et al., 2001). There are several delivery systems to increase quercetin bioavailability: 1) lipid-based carriers; 2) polymer-based carriers or nanoparticles; 3) inclusion complexes; 4) micelles; and 5) conjugates-based capsulations (Wang et al., 2016). One such polymer-based carrier is polyvinylpyrrolidone (PVP). One PVP-based formulation of quercetin provides a 20,000-fold increase in quercetin solubility (Porcu et al., 2018).

CORVITIN® (PJSC SIC “Borshchahivskiy CPP”, Kiev, Ukraine), which combines quercetin with PVP in solid form, is suitable for intravenous injections when dissolved in saline. Quercetin/PVP formulations lower blood pressure in rats both in short-term and long-term bases (Porcu et al., 2018). Prolonged administration (1 month) of CORVITIN® to rabbits following a cholesterol-rich diet significantly decreased atherosclerotic lesion areas in the aorta (Pashevin et al., 2011). CORVITIN® treatment improves cardiac hemodynamics. CORVITIN® treatment also reduces cardiac fibrosis (Kuzmenko et al., 2013).

CORVITIN® administered to patients with acute myocardial infarction decreases the activity of myeloperoxidase in plasma of blood, which is a marker of the metabolic activity of phagocytes and inflammation (Ryzhkova et al., 2016). CORVITIN® treatment results in decreased blood pressure, pulse pressure, improved structural and functional characteristics of the myocardium (including the increase in ejection fraction (EF), and significant decrease of left ventricular end-diastolic dimension (LVEDd), end-diastolic volume (EDV), left ventricular mass index (LVMI), reduced NT-proBNP levels, total NO and improved heart rate variability (Denina, 2013). CORVITIN® is approved in the Ukraine for therapy in patients suffering myocardial infarction and related diseases.

Lyophilization of drugs, particularly biopharmaceuticals, is often used when a drug ingredient is unstable in liquid or frozen form. In addition, lyophilization allows the storage of material for longer periods of time and at room temperature. No studies on the stability of lyophilized quercetin compositions have been performed previously.

### SUMMARY OF THE INVENTION

Provided herein are formulations including a solid form quercetin composition in an enclosed atmosphere consisting essentially of an inert gas or a combination of inert gases. The solid form quercetin composition can be freeze-dried or prepared by spraying drying, rotoevaporation, or crystallization. The inert gas can be essentially oxygen-free. The inert gas can be nitrogen or argon. The solid form quercetin can include a drug delivery formulation. The drug delivery formulation can be a lipid-based carrier, a polymer-based carrier, nanoparticles, inclusion complexes, micelles, or a conjugate-based capsulation. The polymer-based carrier can be polyvinylpyrrolidone (PVP). The solid form quercetin composition can be about 7-11% quercetin and about 89-93% polyvinylpyrrolidone w/w. The solid form quercetin composition can remain greater than 99% of the input quercetin after 24 months at about 20-25° C., or greater than 99% of the input quercetin after 24 months days at about 21° C., or greater than 97% of the input quercetin after 24 months at about 20-25° C., or greater than 97% of the input quercetin after 24 months at about 21° C. The solid form quercetin composition can be less than 0.05% 2,4,6-trihydroxybenzoic acid after 24 months at about 20-25° C., or less than 0.05% 2,4,6-trihydroxybenzoic acid after 24 months at about 21° C., or less than 0.1% of 2,4,6-trihydroxybenzoic acid after 24 months at about 20-25° C., or less than 0.1% of 2,4,6-trihydroxybenzoic acid after 24 months at about 21° C. The solid form quercetin composition after 24 months at 20-25° C. can have a 48 h-EC50 less than 0.5 mg/l in a Daphnia magna mobility assay, or after 24 months at about 21° C. have a 48 h-EC50 less than 0.5 mg/l in a Daphnia magna mobility assay, or after 24 months at about 20-25° C. has a 48 h-EC50 less than 1 mg/l in a Daphnia magna mobility assay, or after 24 months at about 21° C. has a 48 h-EC50 less than 1 mg/l in a Daphnia magna mobility assay.

Provided herein are methods of reducing the rate of formation of toxic byproduct by degradation of a solid form quercetin composition by purging air from an airtight container containing the solid form quercetin composition and filling the container with an atmosphere consisting essentially of an inert gas or a combination of inert gases. The solid form quercetin composition can be freeze-dried or prepared by spraying drying, rotoevaporation, or crystallization. The inert gas can be essentially oxygen-free. The inert gas can be nitrogen or argon. The solid form quercetin composition can include a drug delivery formulation. The drug delivery formulation can be a lipid-based carrier, a polymer-based carrier, nanoparticles, inclusion complexes, micelles, or a conjugate-based capsulation. The polymer-based carrier can be polyvinylpyrrolidone (PVP). The solid form quercetin composition can be 7-11% quercetin and 89-93% polyvinylpyrrolidone w/w. The airtight container can be a glass vial with a stopper and aluminum cap or a glass ampoule.

Provided herein are containers including a solid form quercetin composition and an atmosphere consisting essentially of an inert gas or a combination of inert gases, wherein the container is airtight. The solid form quercetin composition can be freeze-dried or prepared by spraying drying, rotoevaporation, or crystallization. The inert gas can be essentially oxygen-free. The inert gas can be nitrogen or argon. The solid form quercetin composition can include a drug delivery formulation. The drug delivery formulation can be a lipid-based carrier, a polymer-based carrier, nanoparticles, inclusion complexes, micelles, or a conjugate-based capsulation. The polymer-based carrier can be polyvinylpyrrolidone (PVP). The solid form quercetin composition can be 7-11% quercetin and 89-93% polyvinylpyrrolidone w/w. The airtight container can be a glass vial with a stopper and aluminum cap or a glass ampoule.

Provided herein are kits including a plurality of containers of the above in a cassette and an instruction for medical use. The kit can include 5 containers.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows degradation of solid form quercetin composition in air atmosphere under ambient temperature conditions over time;

FIG. 2 shows detection of 2,4,6-THBA by chromatography;

FIG. 3 shows identification of 2,4,6-THBA by mass-spectrometry;

FIG. 4 shows fragmentation of 2,4,6-THBA by mass-spectrometry;

FIG. 5 shows a schema for the mechanism of 2,4,6-THBA formation from quercetin;

FIG. 6 shows UV spectra and molecular structures of 2,4,6-THBA and quercetin;

FIG. 7 shows time-dependent accumulation of 2,4,6-THBA of solid form quercetin composition in air atmosphere under ambient temperature conditions;

FIG. 8 shows effect of substitution of air with nitrogen gas storage atmosphere on degradation of a solid form quercetin composition; and,

FIG. 9 shows effect of substitution of air with nitrogen gas storage atmosphere on 2,4,6-THBA accumulation of a solid form quercetin composition.

### DETAILED DESCRIPTION OF THE INVENTION

Provided herein are compositions and methods which reduce the degradation of quercetin in solid form quercetin compositions and which reduce the formation of toxic compounds, such as 2,4,6-trihydroxybenzoic acid (2,4,6-THBA), that form upon the degradation of solid form quercetin compositions. Also provided are containers that contain solid form quercetin compositions with reduced degradation of quercetin and reduced formation of 2,4,6-THBA. Embodiments of the invention can increase the shelf life and patient safety of solid form quercetin compositions.

Applicant surprisingly found that solid form quercetin compositions undergo degradation under ambient storage conditions. Moreover, Applicant also surprisingly found that at least one of the degradation products is a toxic compound, 2,4,6-trihydroxybenzoic acid (2,4,6-THBA). This toxic byproduct was not predicted by prior studies of quercetin degradation in solution (Wang et al., 2016) Applicant also surprisingly found that by storing solid form quercetin compositions in an inert gas atmosphere, degradation of the product and formation of the toxic byproduct were significantly reduced over a comparable period of time.

Quercetin, 2-(3,4-dihydroxyphenyl)-3,5,7-trihydroxy-4H-chromen-4-one, is a plant flavonoid. Quercetin compositions include relatively pure form quercetin and those that include delivery formulations. Drug delivery refers to approaches, formulations, technologies, and systems for transporting a pharmaceutical compound in the body as needed to safely achieve its desired therapeutic effect. Drug delivery formulations for quercetin can include 1) lipid-based carriers; 2) polymer-based carriers or nanoparticles; 3) inclusion complexes; 4) micelles; and 5) conjugate-based capsulations (Wang et al., 2016). In some embodiments, quercetin compositions include the polymer-based carrier polyvinylpyrrolidone (PVP). In some embodiments, the ratio of PVP:quercetin can be about 8:1, about 9:1, about 10:1, about 11:1, or about 12:1 PVP:quercetin. The relative proportions can vary by about 1%, 2%, 3% or 4%. PVP average molecular weights include, but are not limited to, 8,000, 10,000 or 40,000 g/mol.

In some embodiments, the quercetin composition is in solid form. The solid form of a quercetin composition can be lyophilized (freeze-dried). The solid form of a quercetin composition can be obtained by spray-drying, rotoevaporation or crystallization. When solvent is removed by rotary evaporation, an agglomerated intermediate product is produced, which is then deagglomerated to provide the dry formulation of the quercetin composition. The solid form of a quercetin composition can be provided as a powder, capsules, granules and tablets. In some embodiments, the quercetin composition can be about 9+/−2% quercetin and about 91+/−2% polyvinylpyrrolidone w/w in a lyophilizate.

The solution of the quercetin composition can be sterilized with a sterilizing filter prior to preparing the solid form quercetin composition. Typically, this will involve filtering the solution using a 0.2 micron filter that is solvent compatible, to make a sterile solution. The sterile solution can then be aliquoted directly into dose-sized sterile vials or may be aliquoted at a later time, such as in a sterile fill.

A suitable lyophilization cycle can be readily determined by those skilled in the art, as lyophilization conditions may vary. For example, primary drying conditions may vary from −50° C. to −5° C. The length of the cycle is generally known to those skilled in the art, for example, the cycle length may vary from 8 to 48 hours, generally, sufficient time to remove the solvent or liquid from the product. The secondary drying conditions may vary from 0° C. to 50° C.

Quercetin compositions as a lyophilized powder in an air atmosphere were found to undergo time-dependent degradation with a rate of about 2% per year at room temperature (see Example 2). Storage of solid form quercetin compositions under the same conditions in an inert gas atmosphere have a substantially reduced rate of degradation (see Example 4). In some embodiments, the formulation includes a solid form quercetin composition in a vessel in an atmosphere consisting essentially of an inert gas or a combination of inert gases. An inert gas is a gas that is non-reactive. In some embodiments, the inert gas is nitrogen gas. In some embodiments, the inert gas is argon. In other embodiments, the inert gas is a noble gas. Noble gases include, in addition to argon, helium, neon, krypton, xenon and radon. A combination of inert gases is a plurality of inert gases. Non-limiting examples of a combination of inert gases can be 50% nitrogen/50% argon or 95%/nitrogen/5% argon.

In some embodiments, the formulation comprises a solid form quercetin composition in an atmosphere that is essentially oxygen free.

In some embodiments, the solid form quercetin composition comprises greater than 99% of the input quercetin after 24 months at 15-30° C. In some embodiments, the solid form quercetin composition comprises greater than 99% of the input quercetin after 24 months at about 20-25° C. In some embodiments, the solid form quercetin composition comprises greater than 99% of the input quercetin after 24 months at about 21° C. In some embodiments, the solid form quercetin composition comprises more than 97% of the input quercetin after 24 months at 15-30° C. In some embodiments, the solid form quercetin composition comprises more than 97% of the input quercetin after 24 months at 20-25° C. In some embodiments, the solid form quercetin composition comprises more than 97% of the input quercetin after 24 months at about 21° C. In some embodiments, the solid form quercetin composition comprises more than 97.5%, 98%, or 98.5% of the input quercetin after 24 months at 15-30° C. In some embodiments, the solid form quercetin composition comprises more than 97.5%, 98%, or 98.5% of the input quercetin after 24 months at 20-25° C. In some embodiments, the solid form quercetin composition comprises more than 97.5%, 98%, or 98.5% of the input quercetin after 24 months at about 21° C.

Methods for the detection of quercetin, such as by UV spectroscopy, are known in the art. For example, a solid form quercetin composition is dissolved in 96% v/v analytical grade ethanol and measured spectrophotometrically at 374 nm using 96% ethanol as blank control. In parallel, absorbance of reference solutions (with known quercetin concentrations) are measured. Quercetin content is determined by method of standard.

Quercetin degradation, judging by chromatography, leads to formation of several products. One of the degradation products of solid form quercetin compositions is a toxic compound, 2,4,6-trihydroxybenzoic acid (2,4,6-THBA). This toxic byproduct was not predicted by prior studies of quercetin degradation in solution (Wang et al., 2016).

2,4,6-THBA is toxic to a range of organisms. 2,4,6-THBA has a strong inhibitory effect on cyclin-dependent kinases CDK1, 2, 4, 6 which are key regulators of cell cycle. 2,4,6-THBA exhibits stronger inhibition of purified CDKs in vitro than other salicylic acid metabolites (Dachineni et al., 2017)). 2,4,6-THBA is also more cytotoxic to HCT-116 human cells compared to other salicylic acid metabolites (Dachineni et al., 2017). Thus, 2,4,6-THBA is toxic to mammalian cells when tested in vitro.

Reduction of degradation product of solid form quercetin compositions stored in an inert gas atmosphere results in lowered formation of 2,4,6-THBA (see Example 5).

In some embodiments, the solid form quercetin composition comprises less than 0.05% 2,4,6-trihydroxybenzoic acid after 24 months at 20-25° C. In some embodiments, the solid form quercetin composition comprises less than 0.05% 2,4,6-trihydroxybenzoic acid after 24 months at about 21° C. In some embodiments, the solid form quercetin composition comprises less than 0.1% 2,4,6-trihydroxybenzoic acid after 24 months at 20-25° C. In some embodiments, the solid form quercetin composition comprises less than 0.1% 2,4,6-trihydroxybenzoic acid after 24 months at about 21° C. In some embodiments, the solid form quercetin composition comprises less than 0.06%, 0.07%, 0.08%, 0.09% 2,4,6-trihydroxybenzoic acid after 24 months at about 20-25° C. In some embodiments, the solid form quercetin composition comprises less than 0.06%, 0.07%, 0.08%, 0.09% 2,4,6-trihydroxybenzoic acid after 24 months at about 21° C.

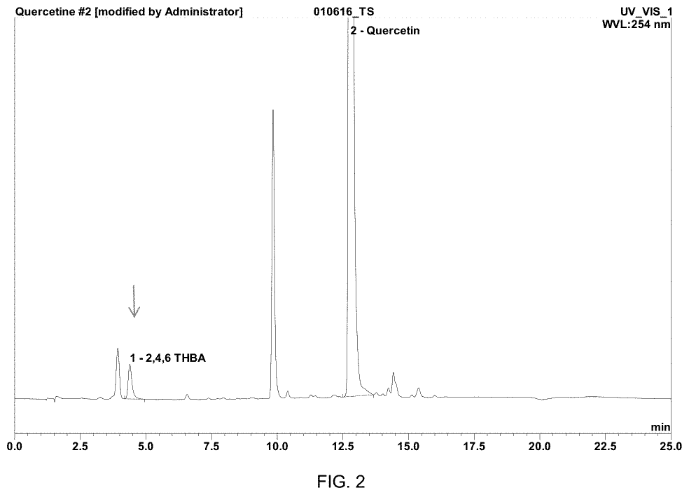

Methods to detect 2,4,6-THBA are known in the art, such as by liquid chromatography with UV detection. For example, a solution containing 2,4,6-THBA is subjected to liquid chromatography using gradient elution with the following conditions: stainless steel column with stationary phase end-capped octadecylsilyl silica for chromatography R; size (150×3.9) mm, particle size 5 micrometers. Mobile phase A: 0.1% phosphoric acid v/v; mobile phase B—methanol; column temperature: 25° C.; flow rate: 1 mL/min.; UV detection at 254 nm. Relative retention time is 1.00 for quercetin, and 0.34 for 2,4,6-trihydroxybenzoic acid (FIG. 2). Content of 2,4,6-trihydroxybenzoic acid relative to quercetin can be calculated by formula (in percentage):

$${X}_{i} = \frac{{S}_{i}}{{S}_{0}} ,$$

where: Sᵢ—peak area of 2,4,6-THBA on the chromatogram of test solution;

S₀—peak area of quercetin on the chromatogram reference solution (a).

Methods to determine toxicity of a compound are known in the art. They include, but are not limited to, in vitro assays for mutagenicity/carcinogenicity (e.g. Ames test in bacteria) and in vitro cytotoxicity (e.g., MTT (e.g. (Dachineni et al., 2017)), XTT, INT or MTS assay, SRB or WST-1 assay in mammalian cells). A label-free approach to follow the cytotoxic response of adherent animal cells is electric cell-substrate impedance sensing (ECIS). Acute and chronic toxicity in rodents and non-human primates can also be used (Parasuraman, 2011). Toxicity of water-soluble compounds can be assessed by appropriate model systems of aquatic organisms such as alga, crustacean, fishes and others (Straub, 2002).

2,4,6-THBA is a metabolite of benzoic acid. As judged by suppression of growth of Pseudokirchneriella subcapitata, a common biological indicator used most extensively by ecotoxicologists, benzoic acid has low toxicity (EC50 is 36 mg/l). In contrast, the EC50 of 2,4,6-THBA is 0.546 mg/l, which is 70-times higher than benzoic acid and its other derivatives (Lee and Chen, 2009). Similarly, benzoic acid demonstrates a low toxicity (860 mg/l EC50 at 48 h), but toxicity of 2,4,6-THBA is about 500 times higher (1.7 mg/l) in a Daphnia magna mobility assays (Kamaya et al., 2005).

Potential human toxicity of 2,4,6-THBA can be related to its toxicity based on studies with Daphnia. Toxicity of various compounds in Daphnia (48 h immobilization test) correlated to toxicity in humans (determined as reference dose for human oral exposure, RfD) (Martins et al., 2007). Given an EC50 of 2,4,6-THBA for Daphnia is 1.7 mg/l, the RfD is estimated to be 0.02 mg/kg/day. For comparison, RfD for 2,4,6-THBA is more than 50-times higher than for benzoic acid (1 mg/kg/day), or 700 times higher than toxicity of quercetin (more than 15 mg/kg/day) in humans (Harwood et al., 2007). 2,4,6-THBA formed as a product of quercetin degradation is therefore toxic to humans.

In some embodiments, the solid form quercetin composition after 24 months at 20-25° C. has a 48 h-EC50 less than 0.5 mg/l in a Daphnia magna mobility assay. In some embodiments, the solid form quercetin composition after 24 months at about 21° C. has a 48 h-EC50 less than about 0.5 mg/l in a Daphnia magna mobility assay. In some embodiments, the solid form quercetin composition after 24 months at 20-25° C. has a 48 h-EC50 less than about 1 mg/l in a Daphnia magna mobility assay. In some embodiments, the solid form quercetin composition after 24 months at about 21° C. has a 48 h-EC50 less than about 1 mg/l in a Daphnia magna mobility assay. In some embodiments, the solid form quercetin composition after 24 months at 20-25° C. has a 48 h-EC50 less than about 0.6, less than about 0.7, less than about 0.8, or less than about 0.09 mg/l in a Daphnia magna mobility assay. In some embodiments, the solid form quercetin composition after 24 months at about 21° C. has a 48 h-EC50 less than about 0.6, less than about 0.7, less than about 0.8, or less than about 0.09 mg/l in a Daphnia magna mobility assay.

Also provided herein are methods of reducing the rate of formation of toxic byproduct by degradation of a solid form quercetin composition by purging air from an airtight container containing the solid form quercetin composition and filling the container with an atmosphere consisting essentially of an inert gas or combination of inert gases. In an embodiment, the method includes displacing the air atmosphere in the container including a solid form quercetin composition with Nitrogen gas. In an embodiment, the method includes displacing the air atmosphere in the container including a solid form quercetin composition with Argon gas.

Also, provided herein are containers that include a solid form quercetin composition and an atmosphere consisting essentially of an inert gas or a combination of inert gases, wherein the container is airtight. Airtight means that gases are not readily exchanged between with inside and outside of the container. Containers include ampoules, vials, syringes, cartridges and bottles. The container can be glass. The glass can be borosilicate glass or soda-lime. The glass can be Type I, II or Ill. The container can be plastic if airtight. The container can be light-safe (e.g. amber). The container can include a stopper, such as a rubber stopper. The stopper can include a septum for introduction of diluent and for removal of solution from the container. Examples of containers include glass vials with a bromobutyl stopper and an aluminum cap.

### EXAMPLES

### Example 1

Preparation of Lyophilized 90%/10% PVP/Quercetin in Nitrogen Atmosphere

Preparation of Intermediate Product Solution

Preparation of alcohol solution (dissolution of quercetin and polyvinylpyrrolidone (PVP) in ethanol) and its evaporation (the formation of a homogeneous dry basis) were carried out with a rotary evaporator (Strike 5000, Steroglass, Perugia, Italy). 25 L Ethanol 96% (high-purity solvent, SE “Ukrspirt”, Lipniki, Ukraine), 1.0 kg quercetin (high-purity solvent, SE “Ukrspirt”, Lipniki, Ukraine) and 9.01 kg polyvinylpyrrolidone (PVP) (EP grade, BASF SE, Ludwigshafen, Germany) were loaded into a 100 liter round-bottomed flask of the rotary evaporator. Dissolution was performed using the following parameters:

| Temperature of water-bath | (70 ± 5) ° C. | |
|---|---|---|
| Vacuum level | 800 | mbar |
| Rate of stirring | 50-100 | rpm |
| Duration | 3.0 | hours |

Stirring continued until the components were completely dissolved (visual control).

Evaporation of alcohol solution (obtaining of dry basis). Upon dissolution, the vacuum level was gradually increased at such a rate to maintain boiling of the solution. According to evaporation of solution the speed of rotation of flask was reduced. The evaporation continued until dryness.

| Temperature of water-bath | (70 ± 5) ° C. | |
|---|---|---|
| Vacuum level at the beginning of | 800 | mbar |
| evaporation | | |
| Vacuum level at the end of | 24-26 | mbar |
| evaporation | | |
| Rate of stirring | 50-100 | rpm |
| Duration of evaporation | (7.0-7.5) | hours |

A sodium hydroxide solution was prepared by charging a reactor (PCBF100, OLSA, Milan, Italy) with 13.5 L water for injection and 41.5 g sodium hydroxide (pharma grade EP, SPOLCHEMIE, Czech Republic) and stirred until complete dissolution. The rotational speed of the mixer was 295-300 rpm and the dissolution time was approximately 5 minutes.

Preparation of Aqueous Solution

The dry basis, obtained at the stage of evaporation of alcohol solution, was dissolved in 52.0 L water for injection. After dissolution of the mass, a reactor (TK001 PCBF50, OLSA, Milan, Italy) was charged with the sodium hydroxide solution using a peristaltic pump (MASTER-FLEX LS 77301-20, MASTER-FLEX, Vernon Hills, USA) to adjust the pH of the solution to about 6.7-7.2. The resulting intermediate product solution was prefiltered using a cartridge filter with a pore size of 0.20 micron (DA36MDMM002MCY2, DANMIL A/S, Greve, Denmark).

Filling of the Vials

The intermediate product solution was tested for microbial load on a filter pursuant to standard methods.

Glass vials (cat #0111075.1063, Medical Glass, Bratislava, Slovak Republics) were filled with a solution of the intermediate product on a filling and capping machine using a sterile filter-capsule with 0.45 and 0.22 micron pore size (SARTOBRAN P, Sartorius Stedim Biotech GmbH, Gottingen, Germany).

The volume of filled intermediate product solution was approximately 3.6-4.2 ml. The filled vials were topped with rubber stoppers (cat # C5919, Aptar Stelmi SAS, Granville, France) in vented position and transferred to a transport laminar trolley (LF 0.6×0.9, CHRIST, Osterode am Harz, Germany) and passed to the lyophilization process.

Lyophilization (Sublimation) of the Intermediate Product Solution

Drying of the intermediate product solution was performed in a lyophilizer (EPSILON 2-45 DS, CHRIST, Osterode am Harz, Germany) according to manufacturer instruction. After lyophilization, the vials were removed from the lyophilizer, nitrogen gas was introduced into each vial using a Nitrogen generator (MAXIGAS108ECALL, PARKER HANNIFIN SP ZOO, Warsaw, Poland), and then the rubber stoppers tightly closed.

Sealing (Packing) of Vials

The stoppered vials were capped with aluminum caps (cat # K-2-20, Chernivets'kyy Zavod Medychnykh Vyrobiv, Chernivtsi, Ukraine) on a filling and capping machine. Sealing (packing), the hermeticity, and the quality of the lyophilized product were tested in accordance with QC procedures.

### Packaging and Labeling

Vial labeling was performed on a labeling machine.

Labeled vials were placed manually into a cassette, 5 vials per cassette. Each cassette together with instruction for medical use was put into a case.

Cases were placed into boxes together with the label “Packer”. The box was covered with adhesive tape (with a logo). A group label printed with the batch number and expiration date was glued with a transparent tape

### Example 2

A Solid Form Quercetin Composition in an Air Atmosphere Undergoes Degradation in Ambient Temperature Storage

Vials of CORVITIN®, a medical formulation of quercetin (10%)/PVP (90%) wherein the solid form quercetin composition is in an air atmosphere, were stored at room temperature (21° C.) and samples were taken at indicated time points (0, 6, 12, 18, 24 and 36 months).

Vial content of quercetin was determined as described below. For test solution, 400.0 mg of vial contents was dissolved in 100 ml 96% (v/v) ethanol. 2.0 ml of the solution was diluted with 96% (v/v) ethanol to 100.0 ml. For reference solution. 40.0 mg of working standard of quercetin (assay: 97.5%-101.5%, PJSC SIC “Borshchahivskiy CPP”, Kiev, Ukraine) was dissolved in 100 ml 96% (v/v) ethanol. Then 2.0 ml of the solution was diluted to 100.0 ml with 96% (v/v) ethanol.

The absorbance of the test solution and of the reference solution was measured using the spectrophotometer at 374 nm and a 1-CM cuvette using 96% (v/v) ethanol as a blank solution.

Vial content of quercetine (X₁) was determined by the formula:

$${X}_{1} = \frac{{A}_{1} * {m}_{0} * 100 * 100 * 2 * b * P}{{A}_{0} * {m}_{1} * 100 * 2 * 100 * 100} = \frac{{A}_{1} * {m}_{0} * b * P}{{A}_{0} * {m}_{1} * 100} ,$$

where A₁—the absorbance of the test solution;
A₀—the absorbance of the reference solution;
m₁—the weight of the sample of the preparation, milligrams;
m₀—the weight of the sample of quercetin reference solution, milligrams;
b—the average vial contents, milligrams;
P— the content of quercetin in quercetin RS, percent.

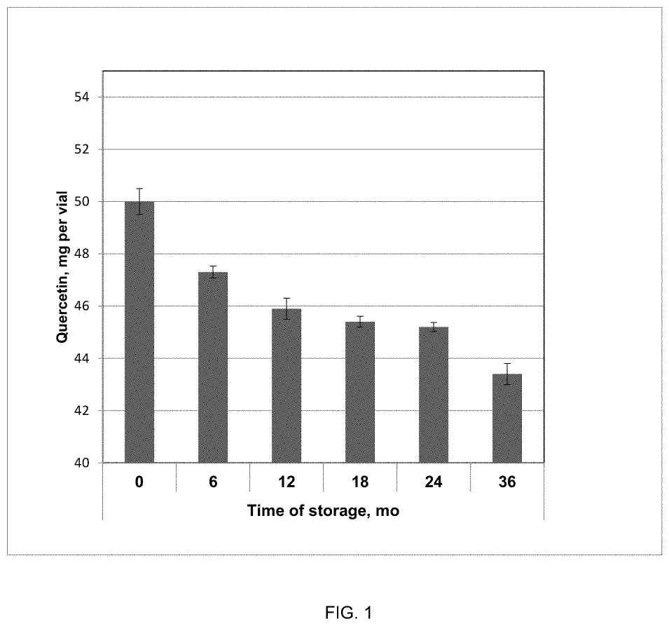

Data from 3 independent experiments are shown in FIG. 1. Based on these experiments, quercetin content in CORVITIN® decreases gradually during storage at a rate of about 2.3% per 12 months

### Example 3

Formation of 2,4,6-trihydroxybenzoic acid (2,4,6-THBA) as a Result of Quercetin Degradation

Vials of CORVITIN® were stored for 18 months at 20-25° C.

The liquid chromatography Dionex UltiMate 3000 with DAD and MS detectors “AB 3200 Q TRAP” was used for analysis. Detectors were connected consecutively: DAD, MS-detector. Ionization ESI and operating mode Q3 and EPI were used. Flavonols, such as quercetin, are easily deprotonated allowing for facile ionization and strong signals at trace amounts in the negative mode. Q3—full scan mode allows to record the MS spectra in a given range (in this case, 50-1000 m/z) at each point of the chromatogram. It also allows establishing the m/z ratio for the molecular ion. EPI (Enhance Product Ion Scan) is used to obtain the mass of fragments; those are formed during the fragmentation of the molecular ion with a given m/z ratio. LIT is used for accumulating fragments to obtain a MS spectra with high-intensity and high resolution. FIG. 2 shows a chromatogram of compounds formed during degradation of quercetin. One of them is identified as 2,4,6-trihydroxybenzoic acid (2,4,6-THBA) by liquid chromatography-mass spectroscopy (LC-MS) as described below.

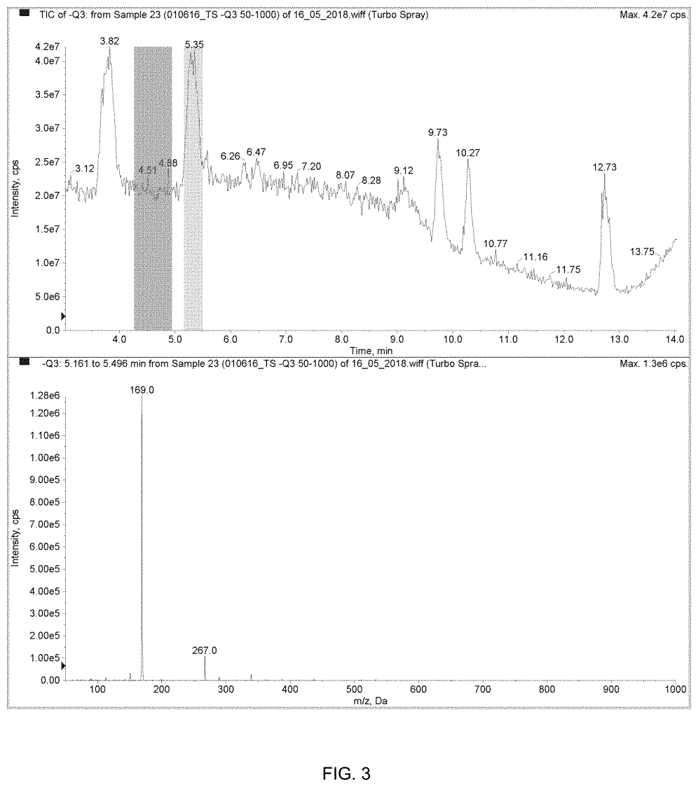

The MS spectrum of the peak with a retention time 5.35 min, (relative retention 0.44) is shown in FIG. 3. The peak with a molecular ion mass m/z=169 and peak of cluster with an unknown compound, that is formed under ionization with m/z=(169+98)=267, were obtained using the Q3 mode.

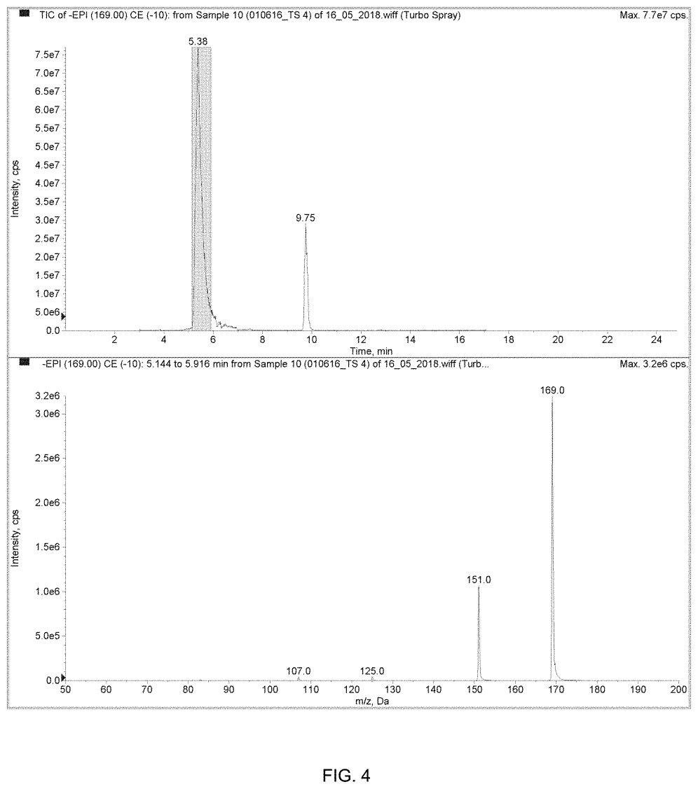

The fragmentation of the molecular ion m/z=169 was determined using EPI mode, negative polarity, with the collision energy—10 V (FIG. 4).

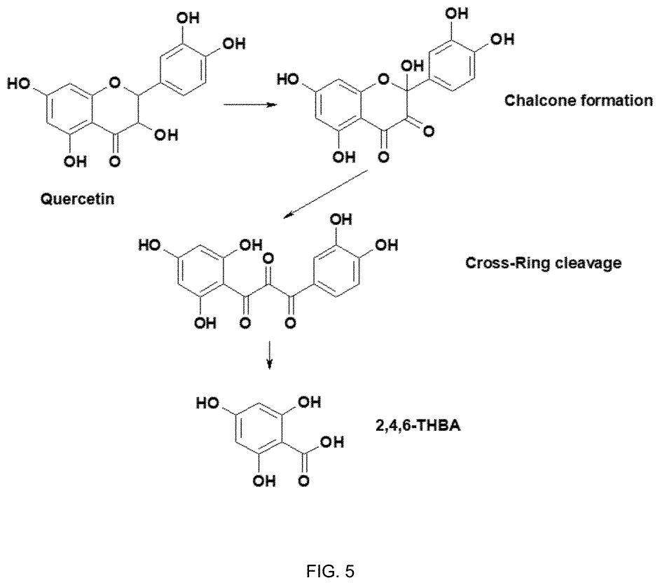

The MS spectra of fragmentation shows that the peak with a relative retention 0.44 corresponds to 2,4,6-trihydroxybenzoic acid. This compound is a product of quercetin degradation through the formation of an intermediate product—chalcone. FIG. 5 shows the proposed chemical mechanism of formation of 2,4,6-THBA during quercetin degradations. During oxidation by air quercetin first forms chalcone which then undergoes cross-ring cleavage forming 2,4,6-THBA as a result.

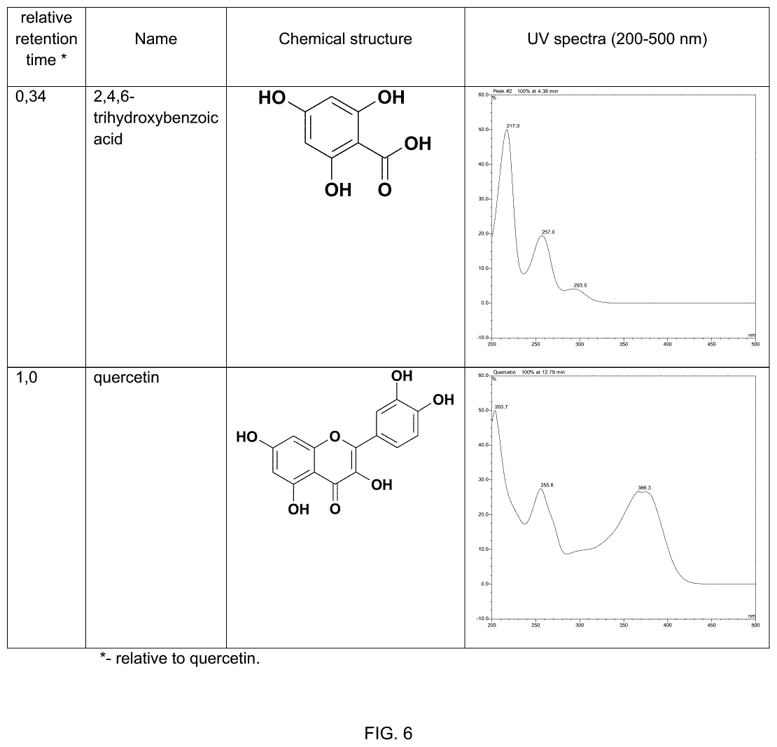

FIG. 6 shows retention times, UV spectra and structures of 2,4,6-trihydroxybenzoic acid (2,4,6-THBA) and quercetin. 2,4,6-trihydroxybenzoic acid has a relative retention time (RRT) 0.34, whereas RRT for quercetin is 1.0. Major absorption peaks of 2,4,6-THBA are 217, 257, and 293.5 nm while those of quercetin are 203.7, 255.6 and 366 nm.

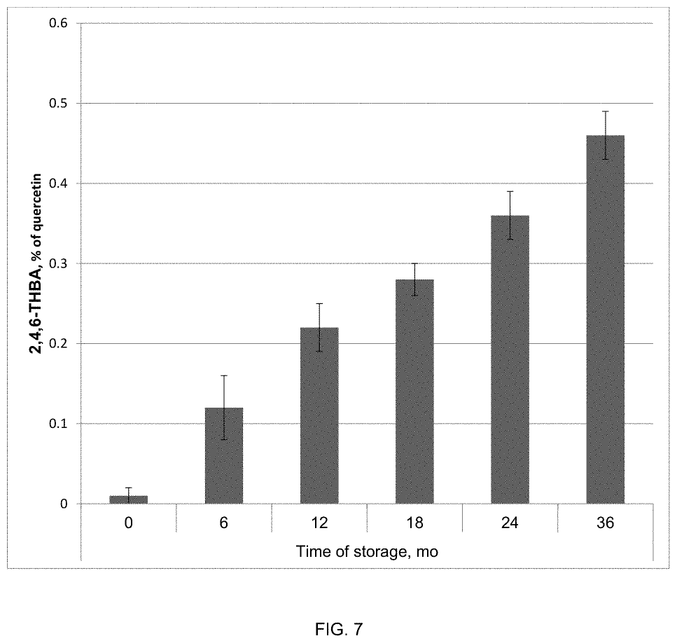

FIG. 7 shows the concomitant time-dependent accumulation of 2,4,6-THBA during quercetin degradation, increasing at each time point measured.

Vials of 10% quercetin/90% PVP were kept at room temperature (21° C.). Samples were removed at indicated time points and assayed for content of 2,4,6-THBA by chromatography as described below. For test solution content of one vial with 70 ml of 96% ethanol was transferred to a 100 ml volumetric flask, diluted to 100 ml with the same solvent and mixed. For reference solution a, 1.0 ml of test solution was placed in a 100 ml volumetric flask, diluted to 100 ml with 96% ethanol and mixed. For reference solution b 10.0 mg working standard of quercetin for system applicability (containing isorhamnetin and kaempferol, PJSC SIC “Borshchahivskiy CPP”, Kiev, Ukraine) was dissolved in 30 ml of 96% ethanol, diluted to 50 ml with the same solvent and mixed. Chromatography was performed on a Dionex liquid chromatograph with UV detector using gradient elution with the following conditions:
stainless steel column with stationary phase end-capped octadecylsilyl silica for chromatography; size (150×3.9) mm, particle size 5 microns
Mobile phase A: 1.0 mL of phosphoric acid diluted to 1000 mL with water for chromatography, mixed and degassed;
Mobile phase B: methanol gradient grade;
Column temperature: 25° C.
Flow rate: 1 mL/min.;
Detection at 254 nm;
injection volume: 10 microL.
Gradient program:

| | Mobile phase A, % | Mobile phase B, % |
|---|---|---|
| Time, min | (V/V) | (V/V) |
| 0 | 80 | 20 |
| 1 | 80 | 20 |
| 16 | 20 | 80 |
| 18 | 20 | 80 |
| 19 | 80 | 20 |
| 25 | 80 | 20 |

Chromatography system was considered to be suitable if the following requirements are performed:
resolution: minimum 2.0 between the principal peak due to quercetin and the peak due to kaempferol.
Inject test solution and reference solution (a).
Peaks of kaempferol and 2,4,6-trihydroxybenzoic acid were determined by relative retention times.

| Name of product | Relative retention |
|---|---|
| 2,4,6-trihydroxybenzoic acid | 0.34 |
| quercetine | 1.00 |
| kaempferol | 1.11 |

Content of 2,4,6-trihydroxybenzoic acid was calculated by formula (in percentages):

$${X}_{i} = \frac{{S}_{i}}{{S}_{0}} ,$$

where: Sᵢ—peak area of the chromatogram of test solution with relative retention 0.34;
S₀—peak area of quercetin on the chromatogram of reference solution (a);
Reporting threshold: 0.05%.

Data from 3 independent experiments are shown. During storage at room temperature, 2,4,6-THBA was gradually accumulated at an approximate rate of 0.15% of quercetin (w/w) per 12 months (FIG. 7).

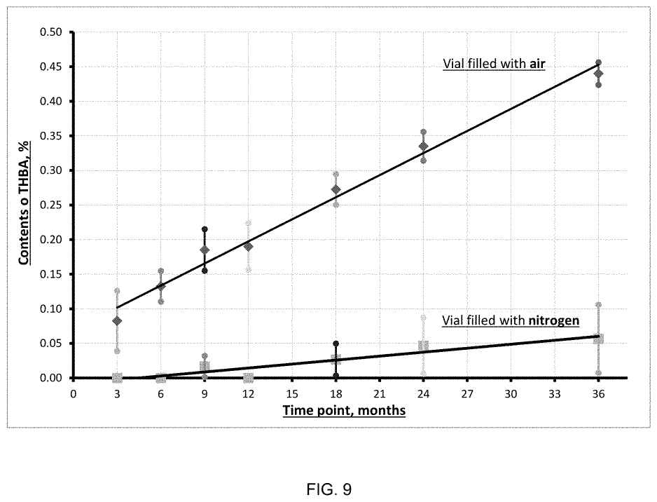

A container of CORVITIN® has 50 mg of quercetin, which is administered i.v. after dissolving with saline to a concentration of 1 mg/ml. Given that the levels 2,4,6-THBA formation after 24 months of quercetin storage under ambient conditions is 0.35% of quercetin content, the 2,4,6-THBA concentration is approximately 3.5 microg/ml, or 3.5 mg/l. Such concentration of 2,4,6-THBA is 2-times higher than the LC50 in 48 h for a Daphna magna immobilization test (1.7 mg/l). Air substitution with nitrogen gas decreases 2,4,6-THBA concentration 7-fold, to 0.05% (FIG. 9) or 0.5 mg/l, which more than 3-times lower than LC50 in 48 h for a Daphna immobilization test (1.7 mg/l). Thus, the amount of 2,4,6-THBA formed from a solid form composition in air during storage conditions is toxic, and substituting air with nitrogen gas reduces 2,4,6-THBA formation to below toxic levels.

### Example 4

Substitution of Air with Nitrogen Gas in Container with Solid Form Quercetin Composition Reduces Quercetin Degradation at Ambient Temperature

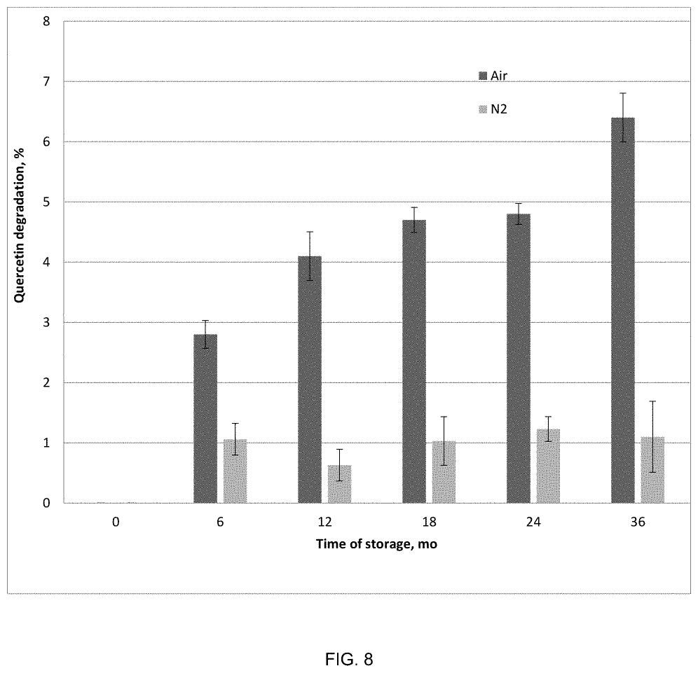

Vials containing (group 1) a lyophilized quercetin (10%)/PVP (90%) composition in an air atmosphere and (group 2) vials containing a lyophilized quercetin (10%)/PVP (90%) composition in a nitrogen gas atmosphere were stored at room temperature. Samples were obtained for each group at the indicated time points (0, 6, 12, 18, 24 and 36 months) and quercetin was assayed by spectrophotometrically at 374 nm and the amount of quercetin remaining calculated as average mass per vial as described above and the percent degradation was calculated for each. Vials containing (group 1) a lyophilized quercetin (10%)/PVP (90%) composition in an air atmosphere showed a significantly greater rate of degradation of quercetin than (group 2) vials containing a lyophilized quercetin (10%)/PVP (90%) composition in a nitrogen gas atmosphere (FIG. 8). Degradation of quercetin was reduced by approximately 80% when the lyophilized quercetin (10%)/PVP (90%) composition was stored in a nitrogen gas atmosphere compared to an air atmosphere at the same ambient storage temperature.

### Example 5

Substitution of Air with Nitrogen Gas in Container with Solid Form Quercetin Composition Reduces Formation of 2,4,6-THBA at Ambient Temperature

Vials containing (group 1) a lyophilized quercetin (10%)/PVP (90%) composition in an air atmosphere and (group 2) vials containing a lyophilized quercetin (10%)/PVP (90%) composition in a nitrogen gas atmosphere were stored at room temperature. Samples were obtained for each group at the indicated time points (0, 3, 6, 9, 12, 18, 24 and 36 months) and assayed for content of 2,4,6-THBA by chromatography as described above. Data from 3 independent experiments are shown. Substitution of air with nitrogen reduced 2,4,6-THBA accumulation by approximately 90% (FIG. 9).

### Example 6

Determination of Toxicity of Solid Form Quercetin Composition Using Inhibition of CDK1 and Cytotoxicity in HCT116 Mammalian Cells

Cytotoxicity is determined by MTT [3-(4,5-dimethylthiazol-2-yl)-2,5-diphenyltetrazolium bromide] assay (Cat. No. 11 465 007 001, Sigma, MO, USA). HCT-116 cells are seeded in 24-well plates overnight at a density of 20,000 cells/well according to manufacturer's instructions. A sample of quercetin (10%) with PVP (90%) is resuspended and introduced at increasing dilutions to the wells. The plates are incubated for 72 h and optical density of formazan product formed is determined as described (Dachineni et al., 2017). The results are compared to similar experiments using benzoic acid and 2,4,6-THBA.

### Example 7

Determination of Toxicity of Solid Form Quercetin Composition Using Pseudokirchneriella subcapitata Growth Assay

Toxicity is determined using green algae Pseudokirchneriella subcapitata growth rate assay as described (Lee and Chen, 2009). Algal inoculum are withdrawn from the chemostat operated under a steady state, and transferred into 300 mL BOD bottles, together with dilution water (with growth medium) and toxicants. The BOD bottles are filled completely, leaving no headspace. A water seal is provided to ensure a closed test environment. The bottles are then placed on an orbital shaker operated at 100 rpm. Temperature and light intensity are kept at 24±1©° C. and 65 microEm−2 s−1 (±10%), respectively. US EPA bottle medium, with no EDTA content is used for toxicity testing. A sample of lyophilized quercetin (10%)/PVP (90%) is resuspended in solution. Two response endpoints are used to evaluate the toxicity of the resuspended quercetin; the final yield and algal growth rate based on cell density counts. The median effective concentration (EC50) is defined as the quercetin composition concentration, which reduces the response to half of that obtained by the control and compared to similar experiments using benzoic acid and 2,4,6-THBA. The initial inoculated cell density is 15,000 cells/mL and the duration of the test is 48 h.

### Example 8

Determination of Toxicity of Solid Form Quercetin Composition Using Daphnia magna Mobility Assay

Toxicity is determined using a Daphnia magna mobility assay as described (Kamaya et al., 2005). Neonates (<24 h old) from 2-3-week-old mothers are placed in a 50 ml glass beaker containing 40 ml of a test solution. All experiments for exposure and controls without chemicals are made in four replicates and performed at 21±0.3° C. under 16 h light: 8 h dark photoperiod. Immobility is used as the endpoint for determining acute toxicity; the daphnids showing no movement within 15 s after gentle stirring are defined to be immobile. After 24 and 48 h, the number of immobile daphnids is recorded to determine the concentration able to achieve 50% immobilization and it is indicated as EC50. The EC50 values are calculated by Probit analyses (USEPA, 1993), based on nominal concentrations and compared to similar experiments using benzoic acid and 2,4,6-THBA.

### EMBODIMENTS

The following are non-limiting embodiments of the invention.

A1. A formulation comprising a solid form quercetin composition in an enclosed atmosphere consisting essentially of an inert gas or a combination of inert gases.

A2. The formulation of embodiment A1, wherein the solid form quercetin composition comprises freeze-dried quercetin.

A3. The formulation of embodiment A1, wherein the solid form quercetin composition is prepared by spraying drying, rotoevaporation, or crystallization.

A4. The formulation of any of embodiments A1-A3, wherein the inert gas is essentially oxygen-free.

A5. The formulation of any of embodiments A1-A4, wherein the inert gas or one of the inert gases of the combination of inert gases is nitrogen.

A6. The formulation of any of embodiments A1-A4, wherein the inert gas or one of the inert gases of the combination of inert gases is argon.

A7. The formulation of any of embodiments A1-A6, wherein the solid form quercetin composition further comprises a drug delivery formulation.

A8. The formulation of embodiment A7, wherein the drug delivery formulation is selected from the group consisting of: a lipid-based carrier, a polymer-based carrier, nanoparticles, inclusion complexes, micelles, and a conjugate-based capsulation.

A9. The formulation of embodiment A8, wherein the drug delivery formulation comprises a polymer-based carrier and the polymer-based carrier comprises polyvinylpyrrolidone.

A10. The formulation of claim A9, wherein the solid form quercetin composition comprises about 8:1, about 9:1, about 10:1, about 11:1, or about 12:1 polyvinylpyrrolidone:quercetin w/w.

A11. The formulation of embodiment A9, wherein the solid form quercetin composition comprises about 7-11% quercetin and about 89-93% polyvinylpyrrolidone w/w.

A12. The formulation of any of embodiments A1-A11, wherein the solid form quercetin composition is stored for 24 months at about 20-25° C.

A13. The formulation of embodiment A12, wherein the solid form quercetin composition is stored for 24 months at about 21° C.

A14. The formulation of any of embodiments A1 to A13, wherein the solid form quercetin composition comprises greater than 97% of the input quercetin.

A15. The formulation of embodiment A14, wherein the solid form quercetin composition comprises greater than 99% of the input quercetin.

A16. The formulation of any of embodiments A1-A15, wherein the solid form quercetin composition comprises less than 0.1% 2,4,6-trihydroxybenzoic acid.

A17. The formulation of embodiment A16, wherein the solid form quercetin composition comprises less than 0.05% 2,4,6-trihydroxybenzoic acid.

A18. The formulation of any of embodiments A1-A17, wherein the solid form quercetin composition has a 48 h-EC50 less than 1 mg/l in a Daphnia magna mobility assay.

A19. The formulation of embodiment A18, wherein the solid form quercetin composition has a 48 h-EC50 less than 0.5 mg/l.

B1. A formulation comprising a solid form quercetin composition, wherein the solid form quercetin composition comprises less than 0.1% 2,4,6-trihydroxybenzoic acid after 24 months storage at about 20-25° C.

B2. The formulation of embodiment B1, wherein the solid form quercetin composition comprises less than 0.1% 2,4,6-trihydroxybenzoic acid after 24 months storage at about 21° C.

B3. The formulation of embodiment B1 or B2, wherein the solid form quercetin composition comprises less than 0.09%, 0.08%, 0.07%, 0.06%, or 0.05% 2,4,6-trihydroxybenzoic acid.

B4. The formulation of any of embodiments B1-B3, wherein the solid form quercetin composition comprises freeze-dried quercetin.

B5. The formulation of any of embodiments B1-B3, wherein the solid form quercetin composition is prepared by spraying drying, rotoevaporation, or crystallization.

B6. The formulation of any of embodiments B1-B5, further comprising an enclosed atmosphere consisting essentially of an inert gas or a combination of inert gases.

B7. The formulation of embodiment B6, wherein the inert gas or combination of inert gases is essentially oxygen-free.

B8. The formulation of embodiment B6, wherein the inert gas or one of the inert gases of the combination of inert gases is nitrogen.

B9. The formulation of embodiment B6, wherein the inert gas or one of the inert gases of the combination of inert gases is argon.

B10. The formulation of any of embodiments B1-B9, wherein the solid form quercetin composition further comprises a drug delivery formulation.

B11. The formulation of embodiment B10, wherein the drug delivery formulation is selected from the group consisting of: a lipid-based carrier, a polymer-based carrier, nanoparticles, inclusion complexes, micelles, and a conjugate-based capsulation.

B12. The formulation of embodiment B11, wherein the drug delivery formulation comprises a polymer-based carrier and the polymer-based carrier comprises polyvinylpyrrolidone.

B13. The formulation of embodiment B12, wherein the solid form quercetin composition comprises about 8:1, about 9:1, about 10:1, about 11:1, or about 12:1 polyvinylpyrrolidone:quercetin w/w.

B14. The formulation of embodiment B12, wherein the solid form quercetin composition comprises about 7-11% quercetin and about 89-93% polyvinylpyrrolidone w/w.

B15. The formulation of any of embodiments B1-B14, wherein the solid form quercetin composition comprises greater than 97% of the input quercetin.

B16. The formulation of embodiment B15, wherein the solid form quercetin composition comprises greater than 99% of the input quercetin.

B17. The formulation of any of embodiments B1-B3, wherein the solid form quercetin composition has a 48 h-EC50 less than 1.0 mg/L in a Daphnia magna mobility assay.

B18. The formulation of embodiment B17, wherein the solid form quercetin has a 48 h-EC50 less than 0.5 mg/L in a Daphnia magna mobility assay.

C1. A container comprising any of the formulations according to embodiments A1-A19 or B1-B18 in a vessel, wherein the container is airtight.

C2. The container of embodiment C1, wherein the airtight container comprises a glass vial with a stopper and aluminum cap.

C3. The container of embodiment C1, wherein the airtight container comprises a glass ampoule.

D1. A kit comprising a plurality of containers according to any of the embodiments C1 to C3 in a cassette.

D2. The kit of embodiment D1 comprising 5 containers.

E1. A method of reducing the rate of formation of a toxic contaminant by degradation of a solid form quercetin composition comprising purging air from an airtight container containing the solid form quercetin composition and filling the container with an atmosphere consisting essentially of an inert gas or a combination of inert gases.

E2. The method of embodiment E1, wherein the solid form quercetin composition comprises freeze-dried quercetin.

E3. The method of embodiment E1, wherein the solid form quercetin composition is prepared by spraying drying, rotoevaporation or crystallization.

E4. The method of any of embodiments E1-E3, wherein the inert gas is essentially oxygen-free.

E5. The method of any of embodiments E1-E4, wherein the inert gas or one of the inert gases of the combination of inert gases is nitrogen.

E6. The method of any of embodiments E1-E4, wherein the inert gas or one of the inert gases of the combination of inert gases is argon.

E7. The method of any of embodiments E1-E6, wherein the solid form quercetin composition further comprises a drug delivery formulation.

E8. The method of embodiment E7, wherein the drug delivery formulation is selected from the group consisting of: a lipid-based carrier, a polymer-based carrier, nanoparticles, inclusion complexes, micelles, and a conjugate-based capsulation.

E9. The method of embodiment E8, wherein the drug delivery formulation comprises a polymer-based carrier and the polymer-based carrier comprises polyvinylpyrrolidone.

E10. The method of embodiment E9, wherein the solid form quercetin composition comprises about 8:1, about 9:1, about 10:1, about 11:1, or about 12:1 polyvinylpyrrolidone:quercetin w/w.

E11. The method of embodiment E9, wherein the solid form quercetin composition comprises 7-11% quercetin and 89-93% polyvinylpyrrolidone w/w

E12. The method of any of embodiments E1-E11, wherein the contaminant comprises 2,4,6-trihydroxybenzoic acid.

E13. The method of any of embodiments E1-E12, wherein the airtight container comprises a glass vial with a stopper and aluminum cap.

E14. The method of any of embodiments E1-E12, wherein the airtight container comprises a glass ampoule.

F1. A method of reducing the degradation of a solid form quercetin composition comprising purging air from an airtight container containing the solid form quercetin composition and filling the container with an atmosphere consisting essentially of an inert gas or a combination of inert gases.

F2. The method of embodiment F1, wherein the solid form quercetin composition comprises freeze-dried quercetin.

F3. The method of embodiment F1, wherein the solid form quercetin composition is prepared by spraying drying, rotoevaporation or crystallization.

F4. The method of any of embodiments F1-F3, wherein the inert gas is essentially oxygen-free.

F5. The method of any of embodiments F1-F4, wherein the inert gas or one of the inert gases of the combination of inert gases is nitrogen.

F6. The method of any of embodiments F1-F4, wherein the inert gas or one of the inert gases of the combination of inert gases is argon.

F7. The method of any of embodiments F1-F6, wherein the solid form quercetin composition further comprises a drug delivery formulation.

F8. The method of embodiment F7, wherein the drug delivery formulation is selected from the group consisting of: a lipid-based carrier, a polymer-based carrier, nanoparticles, inclusion complexes, micelles, and a conjugate-based capsulation.

F9. The method of embodiment F8, wherein the drug delivery formulation comprises a polymer-based carrier and the polymer-based carrier comprises polyvinylpyrrolidone.

F10. The method of embodiment F9, wherein the solid form quercetin composition comprises about 8:1, about 9:1, about 10:1, about 11:1, or about 12:1 polyvinylpyrrolidone:quercetin w/w.

F11. The method of embodiment F9, wherein the solid form quercetin composition comprises 7-11% quercetin and 89-93% polyvinylpyrrolidone w/w.

F12. The method of any of embodiments F1-F11, where in the solid form quercetin composition comprises greater than 97% of the input quercetin after 24 months at about 20-25° C.

F13. The method of embodiment F12, wherein the solid form quercetin composition comprises greater than 97% of the input quercetin after 24 months at about 21° C.

F14. The method of any of embodiments F12 or F13, wherein the solid form quercetin composition comprises greater than 99% of the input quercetin.

F15. The method of any of embodiments F1-F14, wherein the airtight container comprises a glass vial with a stopper and aluminum cap.

F16. The method of any of embodiments F1-F14, wherein the airtight container comprises a glass ampoule.

### REFERENCES

Dachineni, R., D. R. Kumar, E. Callegari, S. S. Kesharwani, R. Sankaranarayanan, T. Seefeldt, H. Tummala, and G. J. Bhat. 2017. Salicylic acid metabolites and derivatives inhibit CDK activity: Novel insights into aspirin's chemopreventive effects against colorectal cancer. International Journal of Oncology 51:1661-1673.
Denina, R. 2013. Heart Failure Treatment In Patients With Recurrent Myocardial Infarction. The Pharma Innovation 2:30-35.
Graefe, E., W. Joerg, M. Silke, R. Anne-Kathrin, U. Bernhard, D. Bernd, P. Holger, J. Gisela, D. Hartmut, and V. Markus. 2001. Pharmacokinetics and Bioavailability of Quercetin Glycosides in Humans. The Journal of Clinical Pharmacology 41:492-499.
Harwood, M., B. Danielewska-Nikiel, J. F. Borzelleca, G. W. Flamm, G. M. Williams, and T. C. Lines. 2007. A critical review of the data related to the safety of quercetin and lack of evidence of in vivo toxicity, including lack of genotoxic/carcinogenic properties. Food and Chemical Toxicology 45:2179-2205.
Kamaya, Y., Y. Fukaya, and K. Suzuki. 2005. Acute toxicity of benzoic acids to the crustacean Daphnia magna. Chemosphere 59:255-261.
Kuzmenko, M. A., V. B. Pavlyuchenko, L. V. Tumanovskaya, V. E. Dosenko, and A. A. Moybenko. 2013. [Experimental therapy of cardiac remodeling with quercetin-containing drugs]. Patol Fiziol Eksp Ter:17-22.
Lee, P. Y., and C. Y. Chen. 2009. Toxicity and quantitative structure-activity relationships of benzoic acids to Pseudokirchneriella subcapitata. Journal of Hazardous Materials 165:156-161.
Martins, J., L. Oliva Teles, and V. Vasconcelos. 2007. Assays with Daphnia magna and Danio rerio as alert systems in aquatic toxicology. Environment International. 33:414-425.
Parasuraman, S. 2011. Toxicological screening. Journal of Pharmacology & Pharmacotherapeutics 2:74-79.
Pashevin, D. A., L. V. Tumanovska, V. E. Dosenko, V. S. Nagibin, V. L. Gurianova, and A. A. Moibenko. 2011. Antiatherogenic effect of quercetin is mediated by proteasome inhibition in the aorta and circulating leukocytes. Pharmacol Rep. 63:1009-18.
Porcu, E. P., M. Cossu, G. Rassu, P. Giunchedi, G. Cerri, J. Pourová, I. Najmanová, T. Migkos, V. Pilařová, L. Nováková, P. Mladěnka, and E. Gavini. 2018. Aqueous injection of quercetin: An approach for confirmation of its direct in vivo cardiovascular effects. International Journal of Pharmaceutics 541:224-233.
Ryzhkova, N. O., T. I. Gavrilenko, and O. M. Parkhomenko. 2016. Korvitin Reduces the High Maintenance of Myeloperoxidase in Plasma of Blood of Patients with the Acute Infarct of Myocardium. Fiziol Zh. 62:87-93.
Straub, J. O. 2002. Environmental risk assessment for new human pharmaceuticals in the European Union according to the draft guideline/discussion paper of January 2001. Toxicology Letters 131:137-143.
Wang, W., C. Sun, L. Mao, P. Ma, F. Liu, J. Yang, and Y. Gao. 2016. The biological activities, chemical stability, metabolism and delivery systems of quercetin: A review. Trends in Food Science & Technology 56:21-38.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

The above disclosure is intended only to convey an understanding of the present invention to those skilled in the art, and is not intended to be limiting. It will be appreciated that various modifications to the disclosed embodiments are possible without departing from the scope of the invention. Therefore, the scope of the present invention should be construed solely by reference to the appended claims.

1. A formulation comprising a freeze-dried quercetin composition in an enclosed atmosphere consisting essentially of an inert gas or a combination of inert gases, said quercetin composition comprising a drug delivery formulation.

2-4. (canceled)

5. The formulation of claim 1, wherein the inert gas or one of the inert gases of the combination of inert gases is nitrogen.

6-7. (canceled)

8. The formulation of claim 1, wherein the drug delivery formulation is selected from the group consisting of: a lipid-based carrier, a polymer-based carrier, nanoparticles, inclusion complexes, micelles, and a conjugate-based capsulation.

9. The formulation of claim 8, wherein the drug delivery formulation comprises a polymer-based carrier and the polymer-based carrier comprises polyvinylpyrrolidone.

10. The formulation of claim 9, wherein the drug delivery formulation comprises about 8:1, about 9:1, about 10:1, about 11:1, or about 12:1 polyvinylpyrrolidone: quercetin w/w.

11. The formulation of claim 9, wherein the drug delivery formulation comprises about 7-11% quercetin and about 89-93% polyvinylpyrrolidone w/w.

12-72. (canceled)

73. The formulation of claim 1, wherein the inert gas is essentially oxygen-free.

74. The formulation of claim 1, wherein the inert gas or one of the inert gases of the combination of inert gases is argon.

75. The formulation of claim 1, wherein the freeze-dried quercetin composition is stored for 24 months at about 20-25° C.

76. The formulation of claim 75, wherein the freeze-dried quercetin composition is stored for 24 months at about 21° C.

77. The formulation of claim 75, wherein the freeze-dried quercetin composition comprises greater than 97% of the input quercetin.

78. The formulation of claim 75, wherein the freeze-dried quercetin composition comprises greater than 99% of the input quercetin.

79. The formulation of claim 75, wherein the freeze-dried quercetin composition comprises less than 0.1% 2,4,6-trihydroxybenzoic acid.

80. The formulation of claim 75, wherein the freeze-dried quercetin composition comprises less than 0.05% 2,4,6-trihydroxybenzoic acid.

81. The formulation of claim 75, wherein the freeze-dried quercetin composition has a 48 h-EC50 less than 1.0 mg/l in a Daphnia magna mobility assay.

82. The formulation of claim 1, wherein the freeze-dried quercetin composition after 24 months at about 21° C. has a 48 h-EC50 less than 0.5 mg/l in a Daphnia magna mobility assay.

83. The formulation of claim 11, wherein the freeze-dried quercetin composition after 24 months at about 20-25° C. comprises less than 0.1% 2,4,6-trihydroxybenzoic acid.

84. The formulation of claim 11, wherein the freeze-dried quercetin composition after 24 months at about 20-25° C. comprises less than 0.05% 2,4,6-trihydroxybenzoic acid.
